(19) United States
(12) Patent Application Publication
(10) Pub. No.: US 2024/0252998 A1
(43) Pub. Date: Aug. 1, 2024

(54) GENERATION OF NANO-BUBBLES IN A LIQUID CARRIER

(71) Applicant: Moleaer, Inc., Hawthorne, CA (US)

(72) Inventor: Federico Pasini, Redondo Beach, CA (US)

(21) Appl. No.: 18/423,987

(22) Filed: Jan. 26, 2024

Related U.S. Application Data
(60) Provisional application No. 63/441,595, filed on Jan. 27, 2023.

(51) Int. Cl. B01F 31/00; A01G 29/00; A01N 25/04; A01N 59/00; A01P 21/00; A23L 2/54; A61K 9/46; B01F 35/221; B01F 35/51; B01F 35/512; B01F 101/00; B01F 101/32; C02F 1/72; C02F 3/02; C02F 101/10; C25B 1/50

(52) U.S. Cl. CPC B01F 31/57; A01G 29/00; A01N 25/04; A01N 59/00; A01P 21/00; A23L 2/54; A61K 9/0007; B01F 35/2211; B01F 35/51; B01F 35/512; C02F 1/727; C02F 3/02; C25B 1/50; B01F 2101/305; B01F 2101/32; B01F 2215/045; B01F 2215/0459; C02F 2101/101; C02F 2303/04

(57) ABSTRACT

Described here are apparatuses and methods for producing a composition comprising nano-bubbles dispersed in a liquid carrier. One such method includes flowing a liquid carrier from an inlet through at least two channels each including a triboelectric material, including flowing the liquid carrier such that a Reynolds number of the flow of the liquid carrier through the at least two channels is less than 3000. Flowing the liquid carrier through the at least two channels produces vibrational energy that causes: (i) the liquid carrier to contact the triboelectric material such that an electric charge is generated in the triboelectric material; and (ii) the liquid carrier to separate from the triboelectric material such that the electric charge is discharged to the liquid carrier to form nano-bubbles dispersed in the liquid carrier.

### CLAIM OF PRIORITY

This application claims priority to U.S. Patent Application Ser. No. 63/441,595, filed on Jan. 27, 2023, the entire contents of which are hereby incorporated by reference.

### TECHNICAL FIELD

This invention relates to apparatuses and methods for producing compositions that include a liquid carrier and nano-bubbles dispersed therein.

### BACKGROUND

Nano-bubbles have several unique properties such as long lifetime in liquid due to their negatively charged surfaces. Nano-bubbles also have high gas solubility into the liquid due to their high internal pressure. Conversely, micro-and macro-bubbles are larger in size, and thus rise rapidly and burst at the water surface.

Nano-bubbles are applicable in variety of fields and can have numerous beneficial effects from a medical, industrial and agricultural point of view. For example, the presence of nano-bubbles can promote a physiological activity in creatures and increase metabolism, as a result of which ontogenetic growth is enhanced.

To date several methods of generating nano-bubbles have been proposed. These methods include swirl-type liquid flow, venturi, high-pressure dissolution, ejector, mixed vapor direct contact condensation and supersonic vibration. All of these methods are energy intensive and have varying degrees of success in creating nano-bubbles.

### SUMMARY

The inventors have discovered that using an apparatus having a particular design, and under particular process parameters, can surprisingly produce high concentrations of nano-bubbles. Without being bound by theory, it is believed that the apparatus described herein, one having conduits of a specific geometry and being composed of at least one triboelectric material, is designed to produce vibrational energy that causes contact between a flowing liquid carrier and the triboelectric material and separation of the flowing liquid from the triboelectric material. The contact between the liquid carrier and the triboelectric material causes an electric charge to be generated in the triboelectric material, and the separation causes the electric charge to be discharged to a volume of the liquid carrier. This process, referred to as liquid-solid contact electrification, results in the formation of nano-bubbles dispersed in the liquid carrier. Because the vibrational energy resulting from the flowing liquid carrier causes generation of nano-bubbles in the liquid carrier (e.g., based on the liquid-solid contact electrification process), the apparatus and methods of using the apparatus advantageously do not require a need for introducing an external gas or the addition of an external energy source when generating the nano-bubbles. Moreover, because the apparatuses and methods described here are capable of generating nano-bubbles without an external gas source or energy source, the apparatuses can be miniaturized apparatuses, e.g., microfluidic apparatuses.

The methods and apparatuses described herein are capable of generating a high concentration of nano-bubbles. In some embodiments, the apparatus can generate nano-bubbles at a concentration of at least 10⁶nano-bubbles per cm³. In some embodiments, the nano-bubble concentration is at least 10⁷nano-bubbles per cm³, at least 10⁸ nano-bubbles per cm³, at least 10⁹nano-bubbles per cm³, at least 10¹⁰nano-bubbles per cm³, or at least 10¹¹ nano-bubbles per cm³. In some embodiments, the composition includes nano-bubbles that are stable in the liquid carrier for at least one month or for at least three months under ambient pressure and temperature.

The nano-bubble concentration is expressed as nano-bubbles per cm³. It is measured by collecting 3 samples from the electrolytic cell (which receives the effluent of the nano-bubble generator) and analyzing each sample within 20 minutes after it has been obtained by Nanoparticle Tracking Analysis using a Nanosight NS3000 analyzer available from Malvern PANalytical. Each sample is filtered using a 0.45 μm filter before it is analyzed using the Nanosight NS3000 analyzer.

In a first aspect, there is described a method for producing a composition comprising nano-bubbles dispersed in a liquid carrier. The method includes flowing a liquid carrier from an inlet through at least two channels each including a triboelectric material, including flowing the liquid carrier such that a Reynolds number of the flow of the liquid carrier through the at least two channels is less than 3000, e.g., less than 2500. Flowing the liquid carrier through the at least two channels at this flow condition produces vibrational energy that causes the liquid carrier to contact the triboelectric material such that an electric charge is generated in the triboelectric material. The produced vibrational energy also causes the liquid carrier to separate from the triboelectric material such that the electric charge is discharged to the liquid carrier to form nano-bubbles dispersed in the liquid carrier.

The liquid carrier flows from the at least two channels to an outlet, where the liquid carrier, now having nano-bubbles dispersed therein, can be recovered.

Flowing the liquid carrier through the at least two channels can include flowing the liquid carrier at a flow rate of at least 3.79×10⁻⁵ m³/min, or at least 3.79×10⁻³ m³/min. For instance, the liquid carrier can be flowed through the at least two channels at a flow rate of between 3.79×10⁻⁵ m³/min and 3.79 m³/min, between 3.79×10⁻³ m³/min and 1.89 m³/min, between 7.57×10⁻³ m²/min and 9.46×10⁻¹ m³/min, or between 1.89×10⁻¹ m³/min and 5.68×10⁻¹ m³/min.

The method can include controlling a flow rate of the liquid carrier through the at least two channels, e.g., using a flow controller disposed between the inlet and the channels. Controlling the flow rate can include, e.g., pulsing the flow of the liquid carrier through the at least two channels.

While the methods described herein do not require the use of an external power source to generate nano-bubbles, the method can involve generating an electric charge in the triboelectric material using an external power source. For instance, the method can include applying a vibration to the triboelectric material using a resonator, or injecting a charge into the triboelectric material from a current source.

These methods generate flow conditions that are sufficient to promote solid-liquid contact electrification. For instance, the flow conditions of the flow of the liquid carrier through the channel are sufficient to produce at least 90 J of vibrational energy.

In some examples, to achieve a Reynolds number of less than 3000, the liquid carrier is flowed at a flow rate of at least 3.79×10⁻⁵ m³/min, in channels having a geometry such that the ratio of an average perimeter of the channels over an average flow path length of the channels is at least about 0.015.

In a second aspect, combinable with the first aspect, there is described an apparatus for producing a composition that includes nano-bubbles dispersed in a liquid carrier. The apparatus includes an inlet for receiving a liquid carrier from a liquid source, an outlet for dispensing a liquid carrier with nano-bubbles dispersed therein, and a housing for generating the nano-bubbles.

The housing includes (a) first and second ends fluidly coupled to the inlet and the outlet, respectively, (b) at least two channels for receiving the liquid carrier from the inlet, wherein the ratio of an average perimeter of the channels over an average flow path length of the channels is at least 0.015, and (c) at least one triboelectric material having an absolute charge density value of at least 15 microcoulomb per square meter (μC/m²).

The apparatus is configured to produce vibrational energy while flowing the liquid carrier through its interior cavity without the use of any external power source or external gas. The geometry of the apparatus is designed such that, when the liquid carrier flows therein at a flow rate of at least 3.79×10⁻⁵ m³/min, the flow of the liquid carrier generates vibrations that cause the liquid carrier to come into contact and subsequently separate from the triboelectric material. When the flowing liquid carrier contacts the triboelectric material, an electric charge is generated in the triboelectric material and can further accumulate in the triboelectric material during the contacting. The vibrations generated by the flow of the liquid carrier also cause the flowing liquid carrier to separate from the triboelectric material such that the accumulated electric charge is discharged to the liquid carrier. The apparatus described herein does not require the introduction of pressurized gas, because the release of the electric charge from the triboelectric material into the liquid carrier results in nano-bubbles forming and dispersing in the liquid carrier. Rather, the vibrations, and the transferred charge, interact with gas that is already dissolved in the liquid carrier to generate the nano-bubbles in the liquid carrier.

The triboelectric material has specific properties, such as an absolute charge density value of at least 15 μC/m2, to promote solid-liquid contact electrification. In some embodiments, the triboelectric material can have absolute charge density value of at least 50 μC/m², of at least 200 μC/m², of at least 500 μC/m², of at least 700 μC/m², or of at least 800 μC/m². The charge density value of the triboelectric material can range from about 15 μC/m²to about 1,200 μC/m², from about 50 μC/m² to about 1,000 μC/m², from about 200 μC/m²to about 950 μC/m², or from about 700 μC/m² to about 900 μC/m². The triboelectric material can carry a negative charge density or a positive charge density.

The triboelectric material can include a polymer. For example, the triboelectric material is PTFE. In some embodiments, the triboelectric material can include polytetrafuoroethylene (PTFE), polyethylene, polyvinyl chloride (PVC), polyethylene terephthalate glycol (PETG), acrylonitrile butadiene styrene (ABS), polylactic acid (PLA), polyamide (PA), ethylene tetrafluoroethylene (ETFE), polymethyl methacrylate (PMMA), or combinations thereof.

The apparatus described herein can include features that are configured to come into contact with the liquid carrier that are made, at least in part, by the triboelectric material. For example, the apparatus can have channels defined by walls, in which at least a portion of the walls include the triboelectric material.

The channels of the apparatus have dimensions designed to promote liquid-solid contact electrification. For example, the ratio of the average perimeter of the channels over the average flow path length of the channels ranges from about 0.015 to about 150, e.g., from about 0.015 to about 30, or from about 0.025 to about 15.

In some cases, at least a portion of the triboelectric material has a surface texture designed to promote liquid-solid contact electrification. For example, the triboelectric material can include a surface texture characterized by a standard deviation of the surface heights distribution (RMS or Rq) of about 0.5×10⁻⁸ meters to 1×10⁻² meters and a mean spacing between profile peaks (Si) of about 0.5×10⁻⁸ meters to 1×10⁻² meters.

The housing of the apparatus includes a plurality of channels. The inlet and/or housing of the apparatus can disperse the liquid carrier into at least two flow paths, in which each flow path flows into a separate channel. Each channel may be in the form of a single channel tube, or a channel of a multi-channel tube. The housing defines at least 2 channels, but may define more than two channels, e.g., at least 10 channels, at least 25 channels, or at least 100 channels. The housing can define from about 2 channels to about 20 channels, from about 5 channels to about 50 channels, or from about 50 channels to about 1,000 channels.

While the apparatus described herein does not require the use of an external power source to generate nano-bubbles, in some embodiments, the apparatus can include an external power source to increase the generation of the electric charge in the triboelectric material. The external power source can add electrical or vibrational energy to the apparatus to increase the generation of the electric charge in the triboelectric material. The external power source can be a resonator (e.g., an electromagnetic resonator or a mechanical resonator), a battery, or both.

The apparatus described herein can include a flow controller, such as a valve, that is configured to control a flow of the liquid carrier through the channels of the apparatus. The flow controller can also include a processor or controller that controls the valve. For instance, the flow controller can control the flow of the liquid carrier so as to generate a flow that promotes liquid-solid contact electrification.

As used herein, the term “nano-bubble” refers to a bubble that has a diameter of less than one micron. A microbubble, which is larger than a nano-bubble, is a bubble that has a diameter greater than or equal to one micron and smaller than 50 microns. A macro-bubble is a bubble that has a diameter greater than or equal to 50 microns.

In a third aspect, combinable with the first or second aspect, there is described a method for producing a composition containing nano-bubbles dispersed in a liquid carrier using the apparatuses described above. The method includes introducing a liquid carrier from a liquid source into the inlet and receiving the liquid carrier from the inlet into the at least two channels of the housing.

In the example method, when the liquid carrier flows through the apparatus, vibrational energy is produced that causes: (i) the liquid carrier to contact the triboelectric material such that an electric charge is generated in the triboelectric material; and (ii) the liquid carrier to separate from the triboelectric material such that the electric charge is discharged to the liquid carrier to form nano-bubbles dispersed in the liquid carrier. In some embodiments, when the liquid carrier is flowing at a flowrate of at least 1×10⁻⁶ cubic meters per minute (m³/min) in the apparatus having a volume of at least 6×10⁻⁷ m³, the apparatus is configured to generate a minimum of about 90 joules (J) of vibrational energy.

The above-described compositions in which nano-bubbles are dispersed in a liquid carrier are useful in a number of applications. For example, the compositions can be used to treat water by transporting the composition to water in need of treatment. Examples of water sources that can be treated include wastewater, oxygen-deficient water, drinking water, and aquaculture water.

In another application, the above-described compositions can be combined with a liquid to create a pumpable composition having a viscosity that is less than the viscosity of the liquid, and then transporting the pumpable composition through a pipe to a desired destination. Examples of liquids include crude oil and drilling or fracking fluids.

In another application, the above-described compositions can be combined with a liquid to create an oxygen-enriched composition, which is then applied to plant roots to enhance plant growth.

In another application, the above-described compositions can be applied in a medical procedure or introduced to a patient using a medical device.

In another application, the above-described compositions can be used to inhibit development of scale in a system, e.g., by circulating the composition in a cooling tower.

In another application, the above-described compositions can be used for electrolysis. For instance, the composition can be used as an electrolyte, or in conjunction with an electrolyte.

The details of one or more embodiments of the invention are set forth in the description below. Other features, objects, and advantages of the invention will be apparent from the description and from the claims.

### BRIEF DESCRIPTION OF DRAWINGS

FIGS. 1-4, 5A-5B, and 6A-6B are diagrams of example apparatuses for producing a composition including nano-bubbles dispersed in a liquid carrier.

FIG. 7 illustrates plots of example surface textures.

FIG. 8 is a flow chart.

### DETAILED DESCRIPTION

We describe here methods and apparatuses for producing a composition including nano-bubbles dispersed in a liquid carrier, under specified flow conditions. Because the vibrational energy produced by the flow of the liquid carrier through the apparatus results in generation of nano-bubbles in the liquid carrier (e.g., based on the aforementioned liquid-solid contact electrification process), the apparatus and methods of using the apparatus advantageously do not require a need for introducing an external gas or the addition of an external energy source when generating the nano-bubbles. Moreover, because the apparatuses and methods described here are capable of generating nano-bubbles without an external gas source or energy source, the apparatuses can be miniaturized apparatuses, e.g., microfluidic apparatuses.

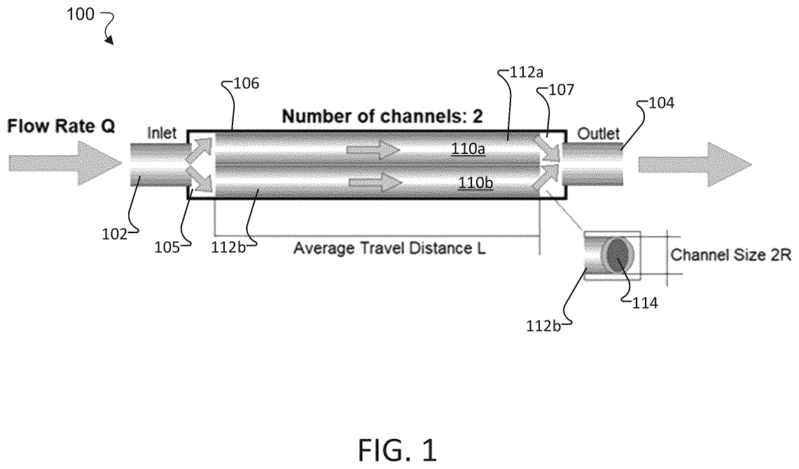

Referring to FIG. 1, an apparatus 100 for producing a composition including nano-bubbles dispersed in a liquid carrier includes an inlet 102 disposed at a first end of a housing 106, and an outlet 104 disposed at an opposite end of the housing 106. In the illustrated example, two flow channels 110a, 110b are defined in an interior of the housing 106, although other numbers of channels can also be used.

The channels 110a, 110b are fluidically coupled to the inlet 102 and positioned to receive liquid carrier from the inlet 102. As the liquid carrier flows into the housing 106 from the inlet 102, the liquid carrier is divided into two flow paths (e.g., as shown by the arrows in FIG. 1) in a distribution chamber 105, with each flow path corresponding to a respective one of the channels 110a, 110b. The liquid carrier flows through the channels 110a, 110b with flow characteristics (e.g., velocity, pressure) that are based on the flow pattern of the liquid carrier through the distribution chamber 105. For instance, the presence of the distribution chamber 105 between the inlet 102 and the channels 110a, 110b distributes the liquid carrier substantially evenly among the channels 110a, 110b, e.g., such that the fluid pressure and flow rate of the liquid carrier is substantially consistent across the channels 110a, 110b.

The liquid carrier exiting the channels 110a, 110b is recombined in an effluent chamber 107 and exits the housing 106 through the outlet 104. As discussed below, the liquid carrier flowing out through the outlet 104 contains a high concentration of nano-bubbles.

In the illustrated example, each channel 110a, 110b is a flow path through a respective single-channel tube 112a, 112b disposed in the interior of the housing 106. In some examples, a single, multi-channel tube is disposed in the interior of the housing 106, and the two channels 110a, 110b are flow paths through a respective channel of the multi-channel tube. In some examples, the channels 110a, 110b are defined by holes extending through a thickness of a solid block of material. Other configurations for the channels 110a, 110b are also possible.

Referring also to the cross-sectional inset in FIG. 1, inner walls 114 of the channels 110a, 110b include a triboelectric material. A triboelectric material is, e.g., a material that is capable of becoming electrically charged upon contact with another material. In the example of FIG. 1, the tubes 112a, 112b themselves are composed of the triboelectric material, e.g., the inner walls 114 of the channels 110a, 110b are the inner walls of the tubes 112a, 112b. In some examples, the triboelectric material is a coating disposed on inner walls of the tubes 112a, 112b, e.g., as a continuous layer of triboelectric material, discontinuous regions of triboelectric material, or another suitable arrangement. All or a portion of the inner walls 114 of the channels 110a, 110b can include the triboelectric material. Other configurations are also possible, e.g., as discussed below.

Flow of the liquid carrier through the channels 110a, 110b from the inlet 102 to the outlet 104 of the housing produces vibrational energy that causes the liquid carrier to come into contact with the inner walls 114 of the channels 110a, 110b, and thus into contact with the triboelectric material, and to separate from the inner walls 114 of the channels 110a, 110b. This contact and separation causes the generation and accumulation of electric charge in the triboelectric material, and the transfer of that electric charge to the liquid carrier, thus forming nano-bubbles in the liquid carrier. Vibrational energy encompasses, e.g., the frequency, amplitude, and acceleration of the relative motion between the liquid carrier and the triboelectric material. For instance, as the liquid carrier flows through the channels 110a, 110b, the vibrational energy generated by that flow causes the liquid carrier to contact the triboelectric material of the inner walls 114 of the channels 110a, 110b. This contact in turn results in generation and accumulation of an electric charge in the triboelectric material. The vibrational energy generated by the flow of liquid carrier through the channels 110a, 110b also causes the liquid carrier to separate from the triboelectric material of the inner walls 114 of the channels 110a, 110b. The separation causes the accumulated electrical charge to be discharged from the surface of the triboelectric material to the liquid carrier, forming nano-bubbles in the liquid carrier. Repeated cycles of contact and separation, and the resulting charge generation and transfer, result in generation of high concentrations of nano-bubbles in the liquid carrier.

Nano-bubbles are generated in the liquid carrier when the liquid carrier flows through the channels 110a, 110b under laminar (e.g., non-turbulent) flow conditions. For instance, nano-bubbles are generated when the Reynolds number of the flow of the liquid carrier through the channels 110a, 110b is less than 3,000, e.g., less than 2,500. Without being bound by theory, it is believed that flow of the liquid carrier under these conditions generates vibrational energy that causes the liquid carrier to contact and separate from the inner walls 114 of the channels 110a, 110b at a frequency and with an intensity (e.g., amplitude) that are sufficient for the generation of nano-bubbles. In an example, the flow of liquid carrier when the Reynolds number of the flow is less than 3,000 or less than 2,500 generates at least about 90 J of vibrational energy.

It is believed that the vibrational energy resulting from velocity and hydraulic pressure gradients during liquid carrier flow through the channels generates mechanical forces at the inner walls 114 of the channels 110a, 110b. These mechanical forces can be converted into electric energy via liquid-solid contact electrification. For instance, when the liquid carrier contacts triboelectric material of the inner walls 114 of the channels 110a, 110b, electric charge is generated in the triboelectric material. When the liquid carrier separates from the triboelectric material, accumulated electric charge is discharged from the triboelectric material to the liquid carrier, resulting in the generation of nano-bubbles in the liquid carrier. The intensity of the discharge of the electric charge depends on the flow of the liquid carrier through the channels 110a, 110b, e.g., on the frequency and the amplitude of the separation of the liquid carrier from the inner walls 114 of the channels 110a, 110b. By flowing the liquid carrier through the channels 110a, 110b under conditions such that the Reynolds number is less than 3,000 or less than 2,500, a frequency and amplitude of the separation can be achieved that are sufficient for generation of high concentrations of nano-bubbles in the liquid carrier.

Flow of the liquid carrier with a Reynolds number of less than 3,000 or less than 2,500 can be achieved by a combination of channel geometry and flow rate of the liquid carrier. For instance, nano-bubbles are generated in the channels 110a, 110b when the geometry (e.g., size) of the channels satisfies a geometric criterion and when the flow rate satisfies a flow rate criterion.

In some examples, the geometric criterion to achieve a Reynolds number of less than 3,000 or less than 2,500, e.g., for flow of a liquid carrier such as water, specifies that a ratio of average perimeter of the channels 110a, 110b in the housing 106 to an average length of the flow paths through the channels 110a, 110b is at least about 0.015, e.g., between about 0.15 and about 150, between about 0.15 and about 30, or between about 0.025 and about 15. We use the term “about” herein to mean within ±10% of the stated value. The perimeter of a given channel is defined as 2π*R, where R is the radius of the channel, and the average perimeter of the channels is the sum of the perimeter of each channel divided by the total number of channels. The length of a given channel is defined as the linear distance between the start and the end of the channel. The average length L of the flow paths is the sum of the length of each channel divided by the total number of channels.

In some examples, the flow rate criterion to achieve a Reynolds number of less than 3,000 or less than 2,500, e.g., for flow of a liquid carrier such as water, specifies that the liquid carrier flow through the apparatus at flow rate of at least 3.79×10⁻⁵ m³/min, e.g., at least 3.79×10⁻³ m³/min, e.g., between 3.79×10⁻⁵ m³/min and 3.79 m³/min, 3.79×10⁻³ m³/min and 1.89 m³/min, between 7.57×10⁻³ m²/min and 9.46×10⁻¹ m³/min, or between 1.89×10⁻¹ m³/min and 5.68×10⁻¹ m³/min.

In a specific example, a Reynolds number of less than 3,000 or less than 2,500 can be achieved when the liquid carrier is flowing at a flowrate of at least 1×10⁻⁶ cubic meters per minute (m³/min) in the apparatus having a volume of at least 6×10⁻⁷ m³. These flow and geometric conditions are sufficient to generate a minimum of about 90 J of vibrational energy, which is believed to be sufficient to enable generation of nano-bubbles.

The triboelectric material has a charge density sufficient to enable generation and transfer of electric charge for nano-bubble formation. For instance, the triboelectric material can have an absolute charge density value (e.g., a positive charge density or a negative charge density) of at least about 15 microcoulomb per square meter (μC/m²), e.g., at least about 50 μC/m², at least about 200 μC/m², at least about 500 μC/m², at least about 700 μC/m², or at least about 800 μC/m², e.g., about 15 μC/m² to about 1,200 μC/m², about 50 μC/m² to about 1,000 μC/m², about 200 μC/m²to about 950 μC/m², or about 700 μC/m² to about 900 μC/m². The charge density of a triboelectric material can be measured, e.g., according to the methods described by Di Liu, et al., “Standardized measurement of dielectric materials' intrinsic triboelectric charge density through the suppression of air breakdown,” Nature Communications (2022) 13:6019, the contents of which are incorporated here by reference in their entirety.

The triboelectric material can be a triboelectric polymer, e.g., polytetrafuoroethylene (PTFE), polyethylene, polyvinyl chloride (PVC), polyethylene terephthalate glycol (PETG), acrylonitrile butadiene styrene (ABS), polylactic acid (PLA), polyamide (PA), ethylene tetrafluoroethylene (ETFE), polymethyl methacrylate (PMMA), high density polyethylene (HDPE), polyvinyl alcohol (PVA), polyethylene terephthalate glycol (PETG), polylactic acid (PLA), polyurethane, polypropylene, acrylic, or another suitable triboelectric material, or a combination of two or more of these materials. The triboelectric material can be a triboelectric glass, ceramic, or carbon fiber. The triboelectric material can be a conductive triboelectric material, such as a metal (e.g., copper, aluminum, stainless steel) or conductive oxide (e.g., titanium oxide or aluminum oxide). In some examples, multiple types of triboelectric material are used within the same apparatus, e.g., multiple types of polymers, multiple types of ceramics, multiple types of metals, or a combination of two or more of polymers, metals, and ceramics. For instance, the triboelectric material can be a ceramic that is coated or doped with an electrode material, such as titanium oxide, aluminum oxide, stainless steel, aluminum, or copper. In some examples, the triboelectric material can be a material, such as a filament material that is suitable for use in an additive manufacturing technique, e.g., engineering PLA filament, ABS filament, PVA filament, PETG filament, nylon filament, carbon fiber filament composite, high impact polystyrene (HIPS), flexible thermoplastic polyurethane filament (TPU), polypropylene filament, or other suitable additively manufactured materials.

The triboelectric material can have a surface texture that promotes contact and separation between the liquid carrier and the triboelectric material as the liquid carrier flows through the channels 110a, 110b. For instance, the triboelectric material has a surface texture characterized by a standard deviation of the surface heights distribution (RMS or Rq) of about 0.5×10⁻⁸ meters to 1×10⁻² meters and a mean spacing between profile peaks (Si) of about 0.5×10⁻⁸ meters to 1×10⁻² meters, as characterized by the methods described by E. S. Gadelmawla, et al., “Roughness parameters,” Journal of Materials Processing Technology (10 Apr. 2002), Vol. 123, No. 1, the contents of which are incorporated here by reference in their entirety. In some examples, the surface texture of the triboelectric material is provided upon formation of the material (e.g., upon deposition of the material, such as in an additive manufacturing process). In some examples, the as-deposited triboelectric material is treated, e.g., in a surface roughening process, to achieve a target surface texture.

Other configurations of flow channels can also be used to generate high concentrations of nano-bubbles by flowing a liquid carrier through the channels under conditions such that the Reynolds number of the flow is less than 3,000 or less than 2,500. For instance, although the channels 110a, 110b of FIG. 1 are shown as linear channels, the channels are not necessarily linear, e.g., the channels can be curved. As another example, although the channels 110a, 110b of FIG. 1 are shown having a circular cross-section, channels with other cross-sectional shapes can also be used. As another example, the channels 110a, 110b can contain one or more internal elements (e.g., a rod disposed within each channel and extending along some or all of the length of the channel) that define additional inner walls including triboelectric material. The channels of the apparatus can have a uniform geometry (e.g., perimeter, length, or cross-sectional shape), or one or more of the channels can have a geometry that differs from the geometry of the other channels.

In some examples, the apparatus 100 can include a port for injection of a gas into the liquid carrier. The gas injection port can be upstream of the channels 110a, 110b. For instance, the port can be for injection of gas into the distribution chamber 105 that is disposed between the inlet port 102 and the channels 110a, 110b. The injection of gas into the liquid carrier can further facilitate nano-bubble generation.

In some examples, an apparatus for producing a composition including nano-bubbles dispersed in a liquid carrier includes more than two flow channels, e.g., at least 10 channels, at least 25 channels, at least 100 channels, or at least 500 channels, e.g., between 2 and 20 channels, between 5 and 50 channels, between 10 and 100 channels, between 100 and 500 channels, or between 500 and 1000 channels. Regardless of the number of channels in the apparatus, nano-bubbles are generated when the flow of the liquid carrier through the apparatus has a Reynolds number of less than 3,000 or less than 2,500, e.g., when the geometry of the channels and the flow rate of the liquid carrier satisfy the geometric and flow rate criteria described above.

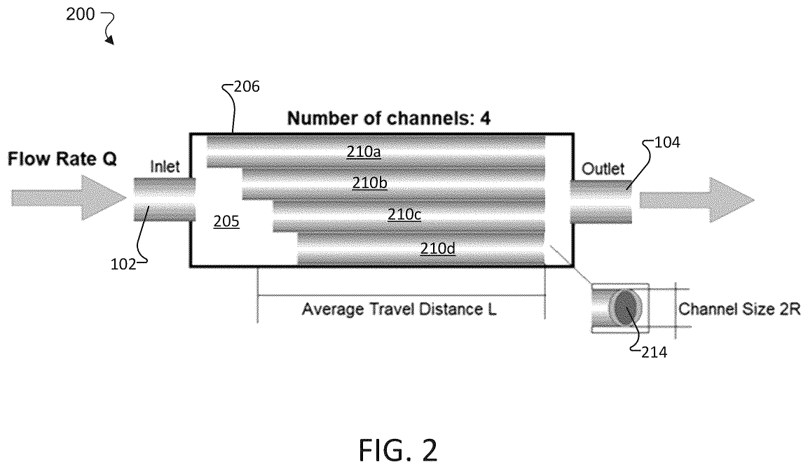

FIG. 2 illustrates an apparatus 200 for producing a composition including nano-bubbles dispersed in a liquid carrier. A housing 206 of the apparatus 200 defines four flow channels 210a-210d, with an inner wall 214 of each flow channel 210a-210d including a triboelectric material. The flow channels 210a-210d are fluidically coupled to an inlet 202 via a distribution chamber 205. The liquid carrier exiting the flow channels 210a-210d, which contains a high concentration of nanobubbles, exits the housing 206 through an outlet 204.

In the example of FIG. 2, the flow channels 210a-210d have different lengths and generally similar perimeters. Nanobubbles are generated in the flow channels 210a-210d of the apparatus 200 when the Reynolds number of the flow of the liquid carrier through the apparatus 200 is less than 3,000 or less than 2,500. For instance, nano-bubbles are generated in the channels 210a-210d when the geometry (e.g., size) of the channels and the flow rate of the liquid carrier satisfy the geometric and flow rate criteria described above. Notably, although the lengths of the individual channels 210a-210d differ in the apparatus 200, the same geometric and flow rate criteria are still applicable.

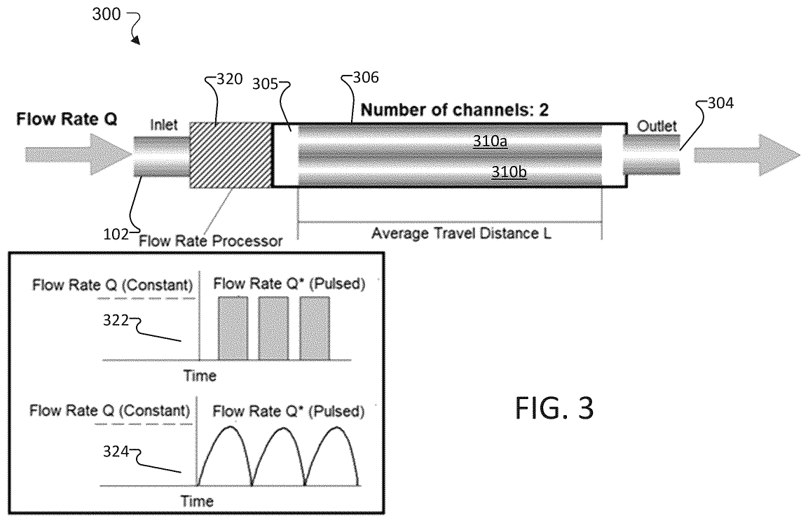

Referring to FIG. 3, an apparatus 300 for producing a composition including nano-bubbles dispersed in a liquid carrier includes a housing 306 defining channels 310a-310b (collectively referred to as channels 310) fluidically connected to an inlet 302 and an outlet 304, e.g., as described above. Nano-bubbles are produced when the Reynolds number of the flow of the liquid carrier through the apparatus 300 is less than 3,000 or less than 2,500, e.g., when the geometry (e.g., size) of the channels and the flow rate of the liquid carrier satisfy the geometric and flow rate criteria described above.

The apparatus 300 includes a flow controller 320, such as one or more valves, e.g., a throttle valve, to control the flow of the liquid carrier in a distribution chamber 305, and thus the flow of the liquid carrier in the channels 310. In the example of FIG. 3, the flow controller 320 is disposed between the inlet 302 and the channels 310. In some examples, the flow controller can be disposed upstream of the inlet 302.

The flow controller 320 can include a processor or controller that controls operation of a valve to generate a time-varying (e.g., pulsed) flow rate, e.g., according to a flow rate waveform such as a square wave 322, a sinusoidal wave 324, a triangular wave, a sawtooth, wave, or another suitable waveform. A time-varying flow of the liquid carrier affects the hydrodynamic conditions of the flow in the channels, which impacts nano-bubble generation, e.g., by enhancing the contact and separation between the liquid carrier and the inner walls of the channels 310. For instance, a time-varying flow of the liquid carrier through the channels 310 results in volumes of liquid carrier flowing through the channels 310, separated by volumes of void, which enhances the contact and separation and thus enhances nano-bubble generation. In some examples, the flow controller 320 can control the direction of the flow, e.g., such that the liquid carrier is provided to a subset of all of the flow channels 310 at any given time.

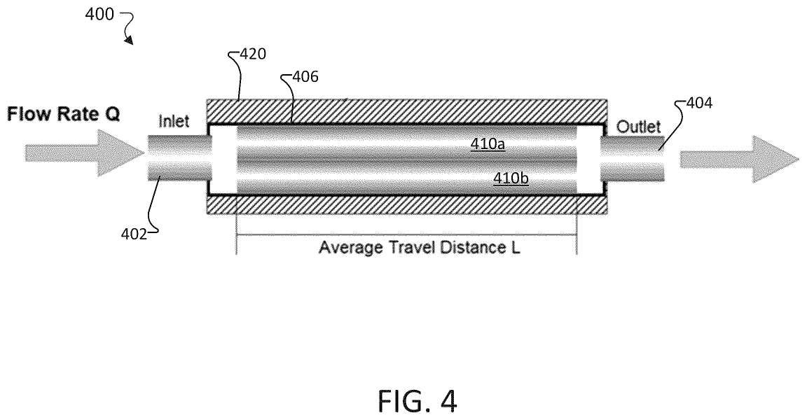

Referring to FIG. 4, an apparatus 400 for producing a composition including nano-bubbles dispersed in a liquid carrier includes a housing 406 housing channels 410a-410b (collectively referred to as channels 410) fluidically connected to an inlet 402 and an outlet 404, e.g., as described above. Nano-bubbles are produced when the Reynolds number of the flow of the liquid carrier through the apparatus 400 is less than 3,000 or less than 2,500, e.g., when the geometry (e.g., size) of the channels and the flow rate of the liquid carrier satisfy the geometric and flow rate criteria described above.

The apparatus 400 includes an external power source 420 that is configured to generate electric charge in the triboelectric material of the channels 410, e.g., to supplement the electric charge generated by the contact between the liquid carrier and the triboelectric material. In some examples, the external power source 420 is a resonator, such as an electromagnetic resonator or a mechanical resonator, that enhances the frequency and amplitude of the contact and separation between the liquid carrier and the inner walls of the channels 410, thereby supplementing the generation of electric charge in the triboelectric material and enhancing the charge transfer to the liquid carrier. For instance, the resonator can be a jacket that fully or partially encircles the housing. In some examples, the external power source is a current source, such as a battery, that is connected to the triboelectric material so as to inject charge into the triboelectric material. In some examples, the external power source is a source of a magnetic field that is configured to induce a current so as to inject charge into the triboelectric material.

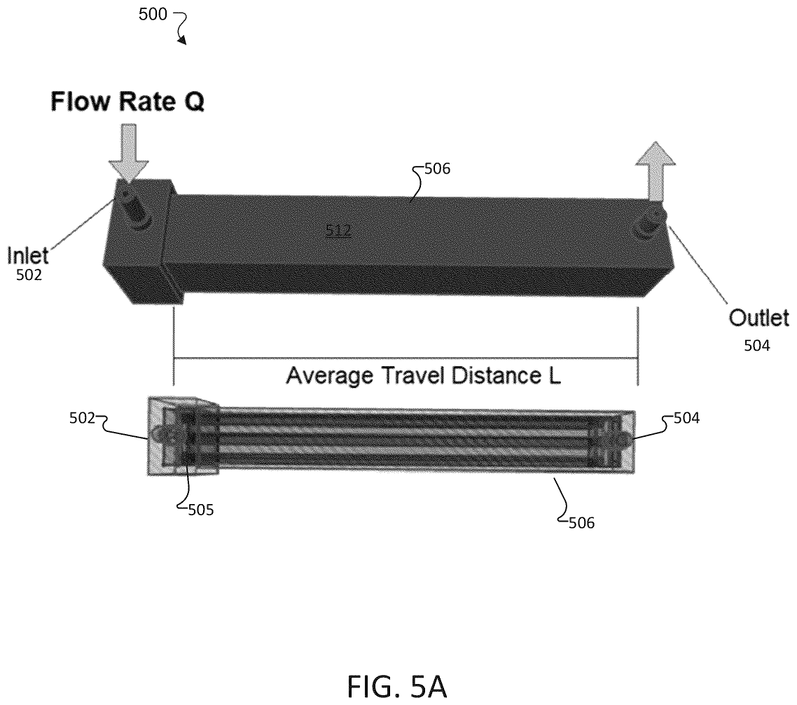

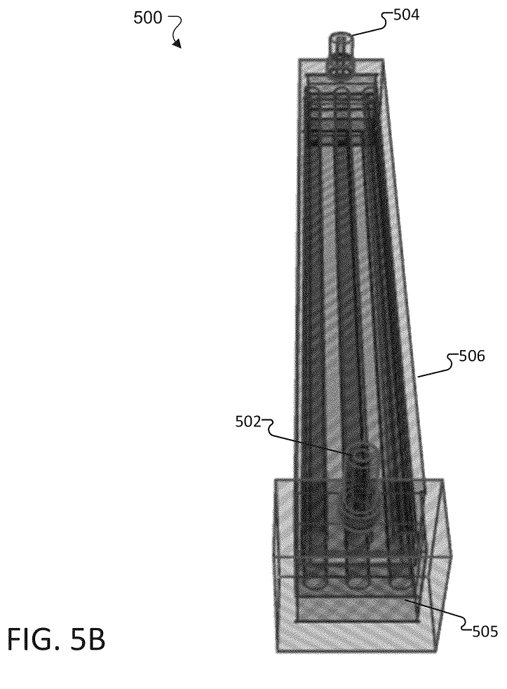

FIGS. 5A and 5B are schematic diagrams of an example apparatus 500 for producing a composition including nano-bubbles dispersed in a liquid carrier. FIG. 5A are top views of the apparatus, and FIG. 5B is a perspective view of the interior of the apparatus. The apparatus 500 includes a housing 506 within which are disposed nine tubes 510 defining channels. The tubes 510 are arranged in a two-dimensional (here, a 3×3) array. The tubes 510 are fluidically connected to an inlet 502 defined in a top wall 512 of the housing 506. Liquid carrier entering the inlet 502 is distributed to the nine tubes 510 in a distribution chamber 505. The tubes are also fluidically connected to an outlet 504 defined in the top wall 512 of the housing. Liquid carrier having a high concentration of nano-bubbles dispersed therein is recovered at the outlet 504. Although in the apparatus 500, the inlet 502 and outlet 504 are defined on the same wall 512 of the housing 506, in some examples, the inlet and outlet can be defined on different walls of the housing 506.

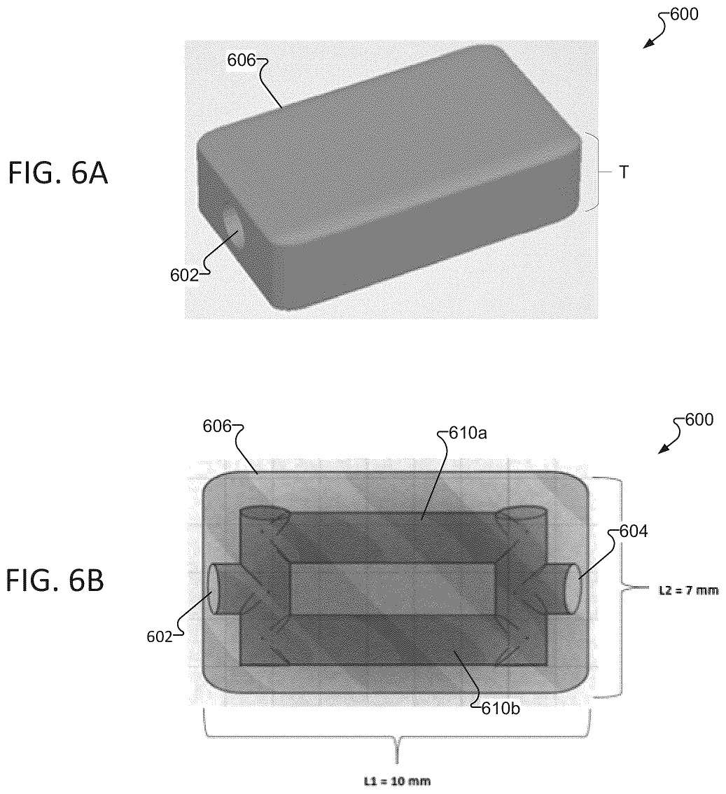

FIGS. 6A and 6B are schematic diagrams of the exterior (FIG. 6A) and interior (FIG. 6B) of an example miniaturized apparatus 600 for producing a composition including nano-bubbles dispersed in a liquid carrier. The apparatus 600 includes an inlet 602 disposed at a first end of a housing 606, and an outlet 604 disposed at an opposite end of the housing 606. In the illustrated example, two flow channels 610a, 610b are defined in an interior of the housing 606, although other numbers of channels can also be used. As discussed above, the use of miniaturized apparatuses for nano-bubble generation is enabled because no external gas source or energy source is necessary for the nano-bubble generation techniques described here.

A miniaturized apparatus such as the apparatus 600 can be used for generation of nano-bubbles when the geometry of the channels and the flow rate of the liquid carrier satisfy the geometric and flow rate criteria described above and when the apparatus satisfies a certain minimum size. For instance, when the apparatus 600 has a length L1 of 10 mm, a width L2 of 7 mm, and a thickness T of 4 mm, and a volume of 2.8×10⁻⁷ m³, and when the geometry of the channels and the flow rate of the liquid carrier satisfy the geometric and flow rate criteria described above, the apparatus is sufficiently sized for nano-bubble generation.

In some examples, multiple of the apparatuses described above can be assembled in series to generate nano-bubbles in larger volumes of liquid carrier. In some examples, multiple of the apparatuses described above can be assembled in parallel to increase the concentration of nano-bubbles generated in the liquid carrier.

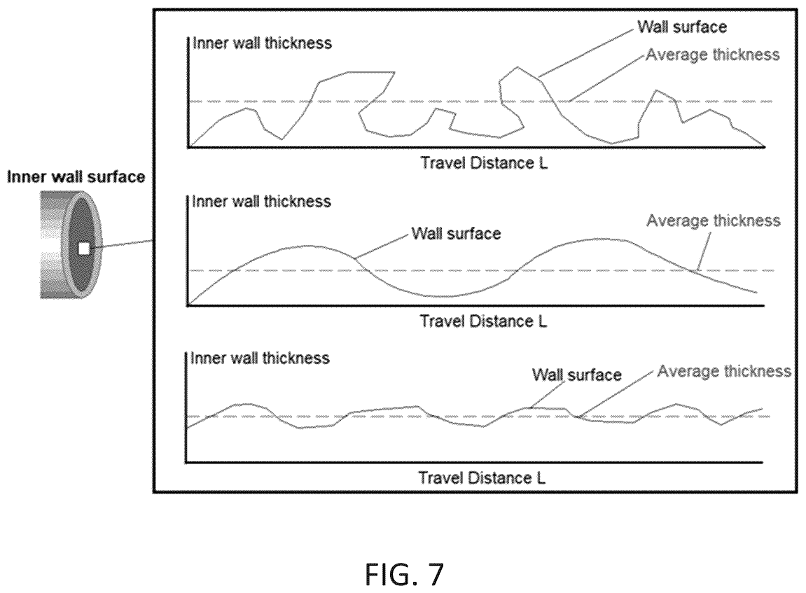

As stated above, the surface texture of the triboelectric material of the inner walls of the channels has a surface texture that promotes contact and separation between the liquid carrier and the triboelectric material as the liquid carrier flows through the channels. FIG. 7 includes plots of example surface texture profiles of the inner walls of channels used for nano-bubble generation, e.g., surface texture profiles that are capable of facilitating the contact and separation dynamics that are relevant for nano-bubble generation. The plots of FIG. 7 plot the thickness of the inner wall of the channel (e.g., the relative thickness of the inner wall relative to the average thickness of the inner wall) versus the length along the channel. The surface texture is characterized by the average thickness of the inner wall, the standard deviation of the thickness, and the rate of variation of the thickness along the length of the channel (e.g., dy/dx, or change in thickness over distance along the channel). As illustrated in the plots of FIG. 7, channels with smooth inner walls (e.g., small standard deviation, small rate of variation, or both) and channels with rougher inner walls (e.g., large standard deviation, large rate of variation, or both) are usable for nano-bubble generation.

In some examples, the material of the inner walls of the channels is selected to generate a contact angle between the liquid carrier and the inner walls that facilitates the contact and separation dynamics that are relevant for nano-bubble generation. For instance, the material of the inner walls can be selected to have a target hydrophobicity, e.g., a target surface energy.

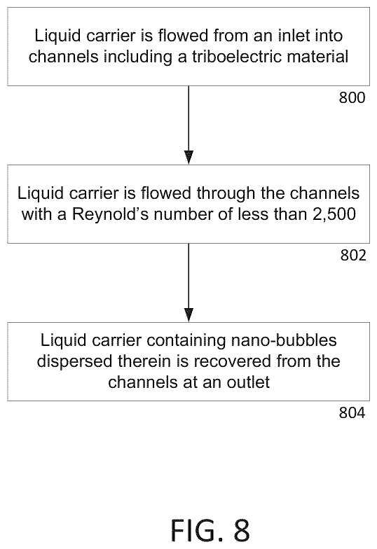

Referring to FIG. 8, in an example method for producing a composition including nano-bubbles dispersed in a liquid carrier, the liquid carrier is flowed from an inlet into at least two channels including a triboelectric material (800). The liquid carrier is flowed through the channels (802) such that a Reynolds number of the flow is less than 3,000 or less than 2,500, e.g., such that the flow is laminar flow. For instance, the liquid carrier is flowed at a flow rate of at least 3.79×10⁻⁵ m³/min, through channels having a geometry such that a ratio of average perimeter of the channels to an average length of the flow paths through the channels is at least about 0.015. The liquid carrier is recovered at an outlet that is downstream of and fluidically connected to the channels (804), where the liquid carrier recovered at the outlet has nano-bubbles dispersed therein.

Nano-bubble containing compositions generated according to the approaches described above are useful in a number of applications. Because the nano-bubbles are stable in the liquid carrier, they may be transported for long distances without dissolving or coalescing in the liquid carrier. Moreover, because the concentration of nano-bubbles in the liquid composition is high, the nano-bubbles are an efficient source for transporting gas to a desired source. In addition, with a smaller surface area and high solubility, compositions containing nano-bubbles are many times more efficient at transferring gases such as oxygen into liquid than conventional aeration.

One application of the nano-bubble containing compositions generated according to the approaches described above involves water treatment. For instance, the composition containing nano-bubbles dispersed in a liquid carrier is transported to a source of water in need of treatment. Examples of water that can be treated include wastewater, oxygen-deficient water, drinking water, and aquaculture water. In the case of drinking water, the nano-bubble containing compositions can be used to create potable water. The nano-bubbles can also be used in carbonated drinking water.

An example water treatment application for these nano-bubble compositions involves environmental water remediation. Because the nano-bubbles having a prolonged lifespan in water and significant mixing potential, the compositions can be used to remediate the ecological balance of surface water, such as lakes, rivers, and the ocean. Enriching water bodies with an abundance of oxygen can help restore beneficial aerobic activity that works to breakdown sludge, hydrogen sulfide, environmental toxins, and pathogenic organisms.

Another application of the nano-bubble containing compositions generated according to the approaches described above involves transporting liquids such as crude oil or drilling fluids through pipes. Often these liquids are viscous and must be transported over significant distances. A composition containing nano-bubbles dispersed in a liquid carrier may be combined with the liquid (e.g., the crude oil, drilling fluid, or fracking fluid) to create a pumpable composition having a viscosity that is less than the viscosity of the liquid to create a pumpable composition that can be transported through a pipe to a desired destination.

Another application of the nano-bubble containing compositions generated according to the approaches described above involves treating plant roots to promote plant growth. For example, a composition containing nano-bubbles dispersed in a liquid carrier can be combined with another liquid to create an oxygen-enriched composition that is then applied to plant roots. Similarly, the compositions containing nano-bubbles in a liquid carrier can be used in aquaculture to create a hyperoxic environment that promotes fish and crustacean growth.

Another application of the nano-bubble containing compositions generated according to the approaches described above involving improving heat transfer. For example, heating or cooling liquids injected with compositions containing nano-bubbles in a liquid carrier can create faster rates of temperature changes in those liquids. A non-limiting exemplary application includes a cooling tower application.

Another application of the nano-bubble containing compositions generated according to the approaches described above involves tissue preservation. Combining the nano-bubble composition with tissue cells can preserve the cells even after freezing.

Another application of the nano-bubble containing compositions generated according to the approaches described above involves vaporization. Compositions containing nano-bubbles dispersed in a liquid carrier have a higher vaporization potential than ordinary water. Thus, combining water in cooling towers with the nano-bubble compositions can enhance the vaporization of cooling tower waters and improve the efficiency of associated cooling processes.

Another application of the nano-bubble containing compositions generated according to the approaches described above involves using the nano-bubble compositions to treat membranes or geothermal wells. When membranes or geothermal wells are continuously exposed to the compositions containing nano-bubbles in a liquid carrier, the compositions can prevent contaminant buildup on the membrane or geothermal well surface. This is due to the fact that the nano-bubbles are negatively charged and can form geometric structures (e.g., lattices) on the membrane or geothermal well surface that exclude certain contaminants, such as salt or organic contaminants.

Another application of the nano-bubble containing compositions generated according to the approaches described above involves using the nano-bubble compositions to reduce or prevent scaling, e.g., in cooling towers.

Another application of the nano-bubble containing compositions generated according to the approaches described above involves using the nano-bubble compositions in the medical field.

Another application of the nano-bubble containing compositions generated according to the approaches described above involves using the nano-bubble compositions in the context of electrolytic processes, e.g., for production of hydrogen, oxygen, chlorine, or other electrolytically produced elements or compounds. For instance, nano-bubble containing compositions can be incorporated into electrolytes for electrolytic processes.

Particular embodiments of the subject matter have been described. Other embodiments are within the scope of the following claims.

What is claimed is:

1. A method for producing a composition comprising nano-bubbles dispersed in a liquid carrier, the method comprising:
flowing a liquid carrier from an inlet through at least two channels each including a triboelectric material, including flowing the liquid carrier such that a Reynolds number of the flow of the liquid carrier through the at least two channels is less than 3000, and
in which flowing the liquid carrier through the at least two channels produces vibrational energy that causes: (i) the liquid carrier to contact the triboelectric material such that an electric charge is generated in the triboelectric material; and (ii) the liquid carrier to separate from the triboelectric material such that the electric charge is discharged to the liquid carrier to form nano-bubbles dispersed in the liquid carrier.

2. The method of claim 1, wherein flowing the liquid carrier through the at least two channels comprises flowing the liquid carrier at a flow rate of at least 3.79×10⁻⁵ m³/min.

3. The method of claim 1, comprising recovering the liquid carrier having nano-bubbles dispersed therein at an outlet, wherein the liquid carrier flows from the channels into the outlet.

4. The method claim 1, comprising controlling a flow rate of the liquid carrier through the at least two channels.

5. The method of claim 4, comprising pulsing the flow of the liquid carrier through the at least two channels.

6. The method of claim 1, comprising generating an electric charge in the triboelectric material using an external power source.

7. The method of claim 6, in which generating the electric charge comprises applying a vibration to the triboelectric material using a resonator.

8. The method of claim 6, in which generating the electric charge comprises injecting a charge into the triboelectric material from a current source.

9. The method of claim 1, in which flowing the liquid carrier through the at least two channels produces at least 90 J of vibrational energy.

10. The method of claim 1, in which flowing the liquid carrier such that the Reynolds number of the flow is less than 3000 comprises flowing the liquid carrier at a flow rate of at least 3.79×10⁻⁵ m³/min, and wherein the ratio of an average perimeter of the channels over an average flow path length of the channels is at least about 0.015.

11. An apparatus for producing a composition comprising nano-bubbles dispersed in a liquid carrier, the apparatus comprising:
an inlet for receiving a liquid carrier from a liquid source;
an outlet; and
a housing comprising: (a) first and second ends fluidly coupled to the inlet and the outlet, respectively; (b) at least two channels for receiving the liquid carrier from the inlet, wherein the ratio of an average perimeter of the channels over an average flow path length of the channels is at least about 0.015, and (c) a triboelectric material having an absolute charge density value of at least 15 microcoulomb per square meter (μC/m²);
wherein, when the liquid carrier flows through the apparatus at a flow rate of at least 3.79×10⁻⁵ m³/min, the apparatus is configured to produce vibrational energy that causes: (i) the liquid carrier to contact the triboelectric material such that an electric charge is generated in the triboelectric material; and (ii) the liquid carrier to separate from the triboelectric material such that the electric charge is discharged to the liquid carrier to form nano-bubbles dispersed in the liquid carrier.

12. The apparatus of claim 11, wherein the absolute charge density value of the triboelectric material is at least 50 μC/m².

13. The apparatus of claim 11, wherein the ratio of the average perimeter of the channels over the average flow path length of the channels ranges from about 0.015 to about 150.

14. The apparatus of claim 11, wherein the triboelectric material comprises a polymer.

15. The apparatus of claim 11, wherein the triboelectric material comprises PTFE.

16. The apparatus of claim 11, wherein the triboelectric material is selected from the group consisting of polytetrafuoroethylene (PTFE), polyethylene, polyvinyl chloride (PVC), polyethylene terephthalate glycol (PETG), acrylonitrile butadiene styrene (ABS), polylactic acid (PLA), polyamide (PA), ethylene tetrafluoroethylene (ETFE), polymethyl methacrylate (PMMA), and combinations thereof.

17. The apparatus of claim 11, wherein the channels are defined by walls that are configured to come into contact with the liquid carrier, and wherein at least a portion of the walls comprise the triboelectric material.

18. The apparatus of claim 11, wherein at least a portion of the triboelectric material has a surface texture characterized by a standard deviation of the surface heights distribution (RMS or Rq) of about 0.5×10⁻⁸ meters to 1×10⁻² meters and a mean spacing between profile peaks (Si) of about 0.5×10⁻⁸ meters to 1×10⁻² meters.

19. The apparatus of claim 11, wherein the inlet, the housing, or both disperse the liquid carrier into at least two flow paths, wherein each flow path flows into a separate channel.

20. The apparatus of claim 11, wherein each channel is a single channel tube.

21. The apparatus of claim 11, wherein the at least two channels are comprised in a multi-channel tube.

22. The apparatus of claim 11, wherein the housing defines at least 2 channels.

23. The apparatus of claim 11, wherein the apparatus comprises an external power source for further generating the electric charge in the triboelectric material.

24. The apparatus of claim 11, wherein the external power source is a resonator, a battery, or both.

25. The apparatus of claim 11, wherein the resonator is an electromagnetic resonator or a mechanical resonator.

26. The apparatus of claim 11, comprising a flow controller configured to control a flow of the liquid carrier through the at least two channels.

27. A method for producing a composition comprising nano-bubbles dispersed in a liquid carrier using the apparatus of claim 11, comprising:
introducing a liquid carrier from a liquid source into the inlet; and
receiving the liquid carrier from the inlet into the at least two channels of the housing;
wherein, when the liquid carrier flows through the apparatus, the apparatus produces vibrational energy that causes: (i) the liquid carrier to contact the triboelectric material such that an electric charge is generated in the triboelectric material; and (ii) the liquid carrier to separate from the triboelectric material such that the electric charge is discharged to the liquid carrier to form nano-bubbles dispersed in the liquid carrier.

28. The method of claim 27, wherein, when the liquid carrier is flowing at a flowrate of between 1×10⁻⁶ cubic meters per minute (m³/min) and 4 m³/min in the apparatus having a flow path volume of between 1.5×10⁻⁷ and 6.5×10⁻¹ m³, the apparatus is configured to generate a minimum of about 90 joules (J) of vibrational energy.

29. A method of treating water comprising:
generating a composition comprising nano-bubbles dispersed in a liquid carrier using the method of claim 1; and
transporting the composition to a source of water in need of treatment.

30. A method of transporting a liquid through a pipe comprising:
generating a composition comprising nano-bubbles dispersed in a liquid carrier using the method of claim 1;
combining the composition with a liquid to create a pumpable composition having a viscosity that is less than the viscosity of the liquid; and
transporting the pumpable composition through a pipe to a desired destination.

31. A method of delivering a liquid to plant roots to promote plant growth, the method comprising:
generating a composition comprising nano-bubbles dispersed in a liquid carrier using the method of claim 1;
combining the composition with a liquid to create an oxygen-enriched composition; and
applying the composition to plant roots to promote plant growth.

32. A method of delivering a liquid during a medical procedure, the method comprising:
generating a composition comprising nano-bubbles dispersed in a liquid carrier using the method of claim 1; and
applying the composition to a medical device or a patient during a medical procedure.

33. A method of regulating temperature in a system, comprising:
generating a composition comprising nano-bubbles dispersed in a liquid carrier using the method of claim 1; and
applying the composition to the system to inhibit development of scale in the system.

34. A method of electrolysis, comprising:
generating a composition comprising nano-bubbles dispersed in a liquid carrier using the method of claim 1; and
performing electrolysis using a fluid containing the composition as an electrolyte.
